(19) United States
(12) Patent Application Publication
(10) Pub. No.: US 2011/0113536 A1
(43) Pub. Date: May 19, 2011

(54) GOGGLES POSITION ADJUSTMENT ASSEMBLIES AND METHODS

(76) Inventors: Jonathan E. Weisel, Norco, CA (US); Anthony Smith, Costa Mesa, CA (US)

(21) Appl. No.: 12/884,030

(22) Filed: Sep. 16, 2010

Related U.S. Application Data
(60) Provisional application No. 61/243,511, filed on Sep. 17, 2009.

(51) Int. Cl. A42B 3/04; A61F 9/02; A42B 3/18

(52) U.S. Cl. 2/422; 2/10

(57) ABSTRACT

An assembly for adjustment of the position of goggles with respect to a wearer's face can comprise a strap engagement member that is movable between a first position and a second position. When in the first position, the strap engagement member can be positioned or oriented to permit the goggles to rest against the face of a wearer. In the second position, the strap engagement member can be positioned or oriented so as to hold the goggles away from the face of the wearer.

### CROSS-REFERENCE TO RELATED APPLICATIONS

This application claims the benefit of U.S. Provisional Patent Application No. 61/243,511, filed on Sep. 17, 2009, which is hereby incorporated by reference herein in its entirety.

### FIELD OF THE DISCLOSURE

The present application is directed generally to devices and methods for positioning goggles relative to a wearer's face.

### BACKGROUND

In some instances, moisture can become trapped between goggles and a wearer's face. Under some conditions this moisture can condense on the optical portion of the goggles, obscuring the wearer's vision. To avoid such fogging of the optical portion of the goggles, it may be desirable to space the goggles from the wearer's face to allow air to flow between the goggles and the wearer's face to release the moisture.

### SUMMARY OF THE DISCLOSURE

In some instances, a wearer of goggles may desire air to flow between the goggles and the wearer's face without completely removing the goggles from the wearer's face and/or head, and thereafter quickly and easily restore the goggles to its former position. Such instances may include, for example, during participation in sporting activities such as motorcross, skiing, and snowboarding. In some embodiments, the assembly can be movably mounted to a helmet 102, such as a sports helmet, e.g., a motorcycle helmet.

In some embodiments, an assembly for adjustment of the position of goggles with respect to a wearer's face can comprise a strap engagement member that is movable between a first position and a second position. When in the first position, the strap engagement member can be positioned or oriented to permit the goggles to rest against the face of a wearer. For example, when in the first position, the strap engagement member can be in close proximity to the wearer's head or to an external surface of the helmet 102, if so mounted. In the second position, the strap engagement member can be positioned or oriented so as to hold the goggles away from the face of the wearer. For example, in the second position, at least a portion of the strap engagement member can be advanced forward a distance that is sufficient to hold the goggles away from the face of the wearer. The distance that portion of the strap engagement member is advanced may be determined with reference to a distance from a wearer's face, an opening in the helmet (if the assembly is so mounted), and/or a location of the connection of the goggles to a strap when the goggles are against the wearer's face.

In some embodiments, the goggles position adjustment assembly can retain the goggles in the second position such that the goggles remains spaced away from the wearer's face until affirmatively moved to the first position.

The distance between the goggles and the wearer's face may be, for example, at least ¼ inch, at least ½ inch, at least 1 inch, at least 1.5 inches, at least 2 inches, at least 2.5 inches, or at least 3 inches.

### BRIEF DESCRIPTION OF THE DRAWINGS

FIG. 1 is a top perspective view of a goggles position adjustment device attached to a helmet and positioning the goggles forward of a typical use position, according to an embodiment.

FIG. 2 is a top plan view of the goggles position adjustment device of FIG. 1 in a retracted configuration.

FIG. 3 is a lower perspective view of the goggles position adjustment device of FIG. 1 in the retracted configuration, also illustrated in FIG. 2.

FIG. 4 is a top plan view of the goggles position adjustment device of FIG. 1 in an extended configuration.

FIG. 5 is a lower perspective view of the goggles position adjustment device of FIG. 1 in the extended configuration, also illustrated in FIG. 4.

FIG. 6 is a front and side perspective view of the goggles position adjustment device attached to a helmet as in FIG. 1 and in the retracted configuration, also illustrated in FIGS. 2 and 3.

FIG. 7 is a front and side perspective view of the goggles position adjustment device attached to a helmet as in FIG. 1 and in an extended configuration.

FIG. 8 is a top-left perspective view of the goggles position adjustment device attached to a helmet as in FIG. 1 and in the retracted configuration, also illustrated in FIGS. 2, 3, and 6, permitting the goggles to rest in a typical use position.

FIG. 9 is a left side view of the goggles position adjustment device attached to a helmet as in FIG. 1 and in the retracted configuration, permitting the goggles to rest in a typical use position against a wearer's face.

FIG. 10 is a left side view of the goggles position adjustment device attached to a helmet as in FIG. 1 and in an extended configuration to position the goggles away from the wearer's face.

FIG. 11 is a partial-cross-sectional top view of the goggles position adjustment device attached to a helmet as in FIG. 1 and in the retracted configuration, permitting the goggles to rest in a typical use position against a wearer's face.

FIG. 12 is a partial-cross-sectional top view of the goggles position adjustment device attached to a helmet as in FIG. 1 and in an extended configuration to position the goggles away from the wearer's face.

FIG. 13 is a left side plan view of an goggles position adjustment device according to an embodiment with an arm in a forwardly and outwardly extending configuration.

### DETAILED DESCRIPTION OF THE EXEMPLIFYING EMBODIMENTS

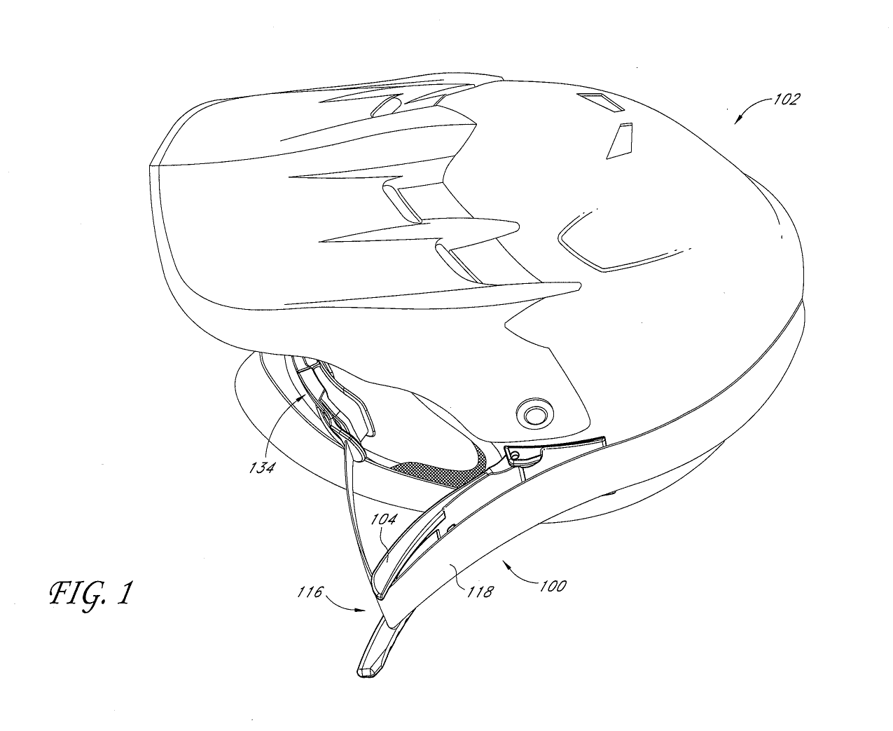

FIG. 1 shows an embodiment of a goggles position adjustment assembly 100 according to an embodiment. The embodiment of FIG. 1 is shown attached to a motorcycle helmet 102. In other embodiments, the goggles position adjustment assembly can be attached to other types of helmets. For example, in some embodiments, the position adjustment assembly can be attached to helmets for skiing, snowboarding, kayaking, biking, climbing, skating, skateboarding, and helmets or other headgear for participating in other activities. In some embodiments, a goggle position adjustment assembly can be used independent of any helmet.

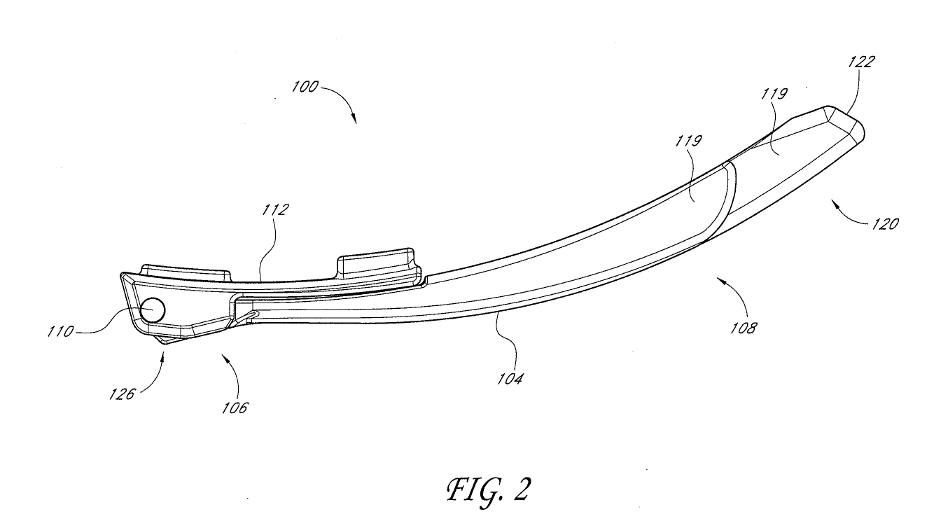

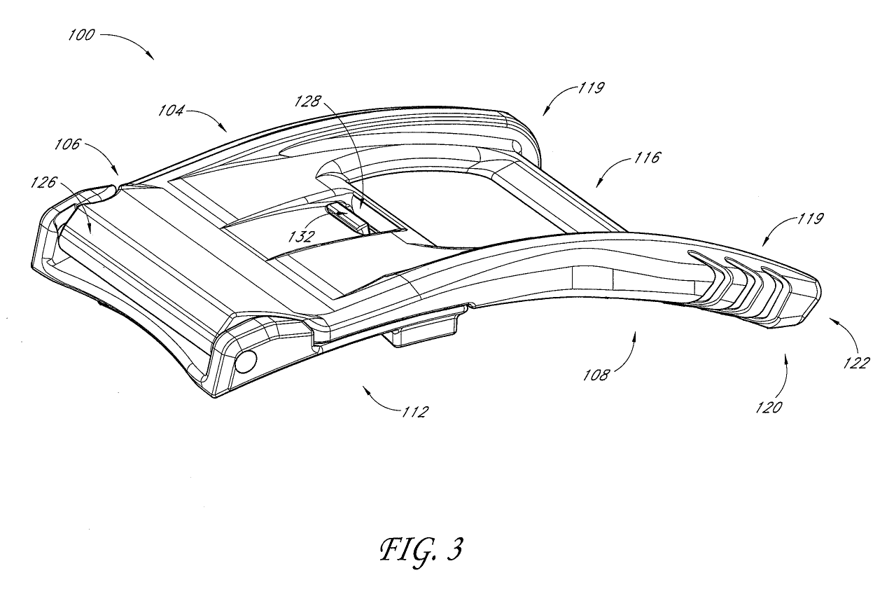

FIGS. 2 and 3 are top and perspective views, respectively, of the goggles position adjustment assembly 100, shown in FIG. 1.

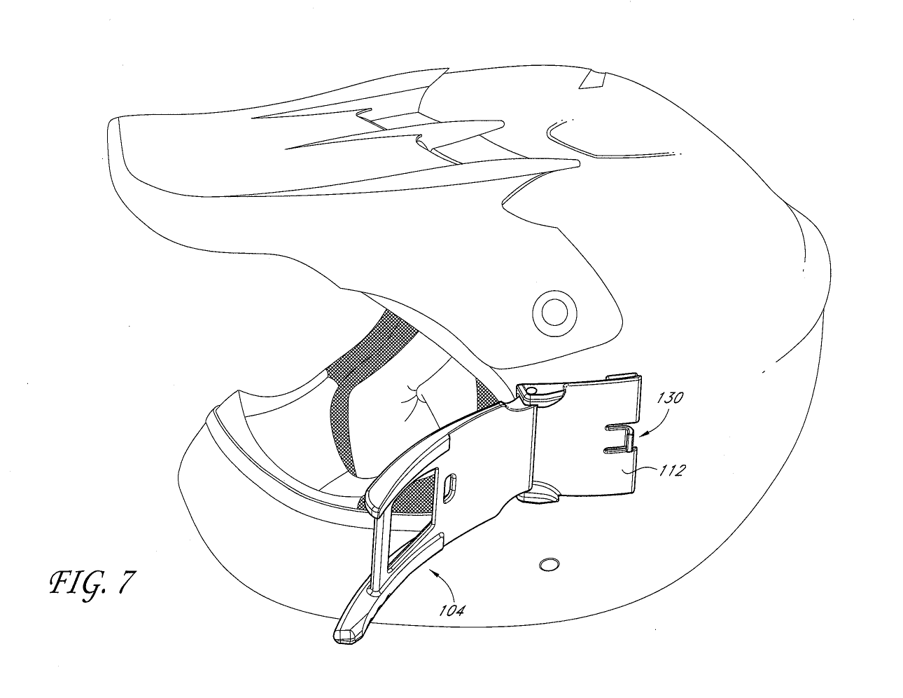

In some embodiments, the strap engagement member can comprise an arm 104, as shown in FIGS. 1-3, for example. The arm 104 can comprise a first end 106 and a second end 108. The arm 104 can be movably attached to the helmet 102. In some embodiments, the first end 106 of the arm 104 can be pivotally attached to the helmet 102, for example, by a hinge 110. The hinge 110 (if present) or other connection can allow the arm 104 to pivot between the first position (FIGS. 1, 4, 5, 7, 10, and 11) and the second position (FIGS. 2, 3, 6, 8, 9, and 12). In some embodiments, the arm 104 can pivot about the hinge through an angle of at least 45°, 90°, approximately 90°, more than 90°, at least 95°, at least 100°, at least 110°, at least 120°, at least 130°, at least 135°. The hinge can comprise a pin or pins in operative engagement with corresponding socket(s), as in the illustrated embodiments. The arm 104 can be coupled to the helmet by other forms of attachment that permit pivoting or rotation between a first position and a second position of the arm.

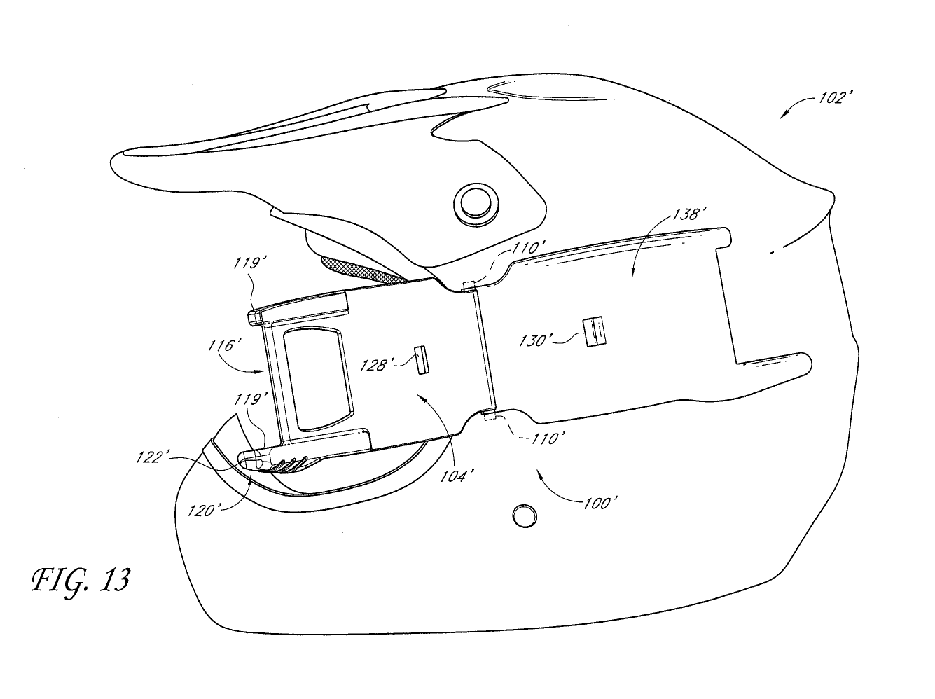

In some embodiments, the strap engagement member can comprise a mounting plate 112. The first end 106 of the arm 104 can be attached by the hinge 110, for example, to the mounting plate 112 in some embodiments. In other embodiments, the arm 104 can be attached by the hinge 110 directly to a side 114 of the helmet 102, for example as illustrated in FIG. 13. In some embodiments, the mounting plate 112 can be attached to a side 114 of the helmet 102, for example as illustrated in FIG. 1. In some embodiments, the mounting plate can be attached to the side 114 of the helmet 102 by an adhesive or suitable fastener, such as, for example, glues, epoxy, adhesive tape, bolts, screws, and rivets.

In some embodiments, the strap engagement member can be attached to the left side of a helmet 102, as illustrated in FIGS. 1 and 6-13. In some embodiments, mounting of the strap engagement member on the left side of the helmet can advantageously allow the wearer to actuation the goggles position adjustment assembly 100 with a left hand while otherwise using the right hand. For example, if the wearer is operating a motorcycle, the right hand can operate a throttle, brake, or both of the motorcycle with the right hand while adjusting the assembly 100 with the left hand.

The strap engagement member can be attached to the right side of the helmet is some embodiments. Also, in some embodiments, strap engagement members can be attached to the right and left sides of a helmet.

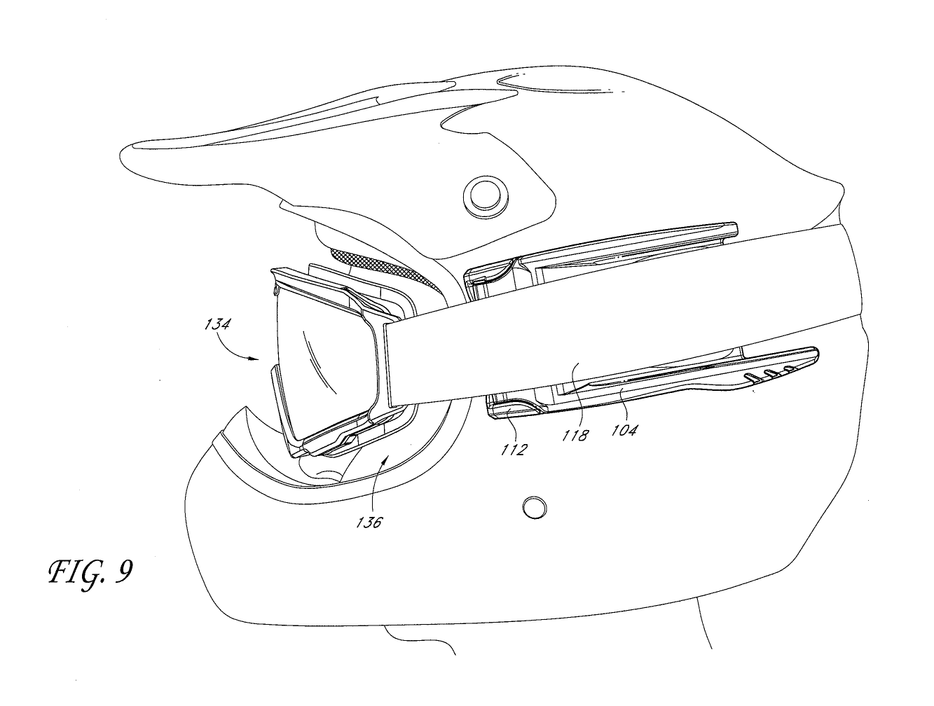

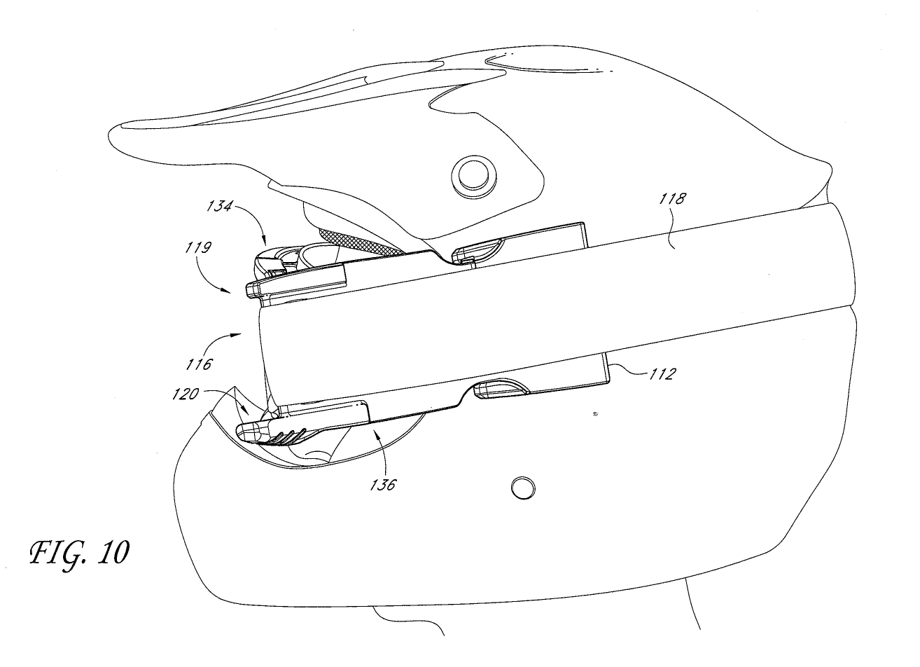

The second end 108 of the arm 104 can comprise a recess 116 to retain the strap 118 in operative engagement with the strap engagement member. The recess 116 can face away from the first end 106 of the strap engagement member, as shown in the illustrated embodiments. The recess 16 can, however, face other directions, such as upwardly, generally upwardly, downwardly, or generally downwardly (with reference to the orientation of the strap engagement member in FIGS. 9 and 10) in some embodiments. In some embodiments, the second end 108 can comprise a hole or opening (not shown) through which the strap extends to retain the strap in operative engagement with the strap engagement member. In some embodiments, the second end 108 of the arm 104 can comprise one or more projections 119, as illustrated in FIG. 1-13, for example. In some embodiments, one or more projections 119 can be positioned above or below the strap 118, or both, to retain the strap 118 in operative engagement with the strap engagement member, as illustrated in FIGS. 1 and 8-10, for example. In some embodiments, the spacing between an upper projection 119 and a lower projection 119 can be at least as great as a width of the strap 118, as illustrated in FIGS. 1 and 8-10, for example.

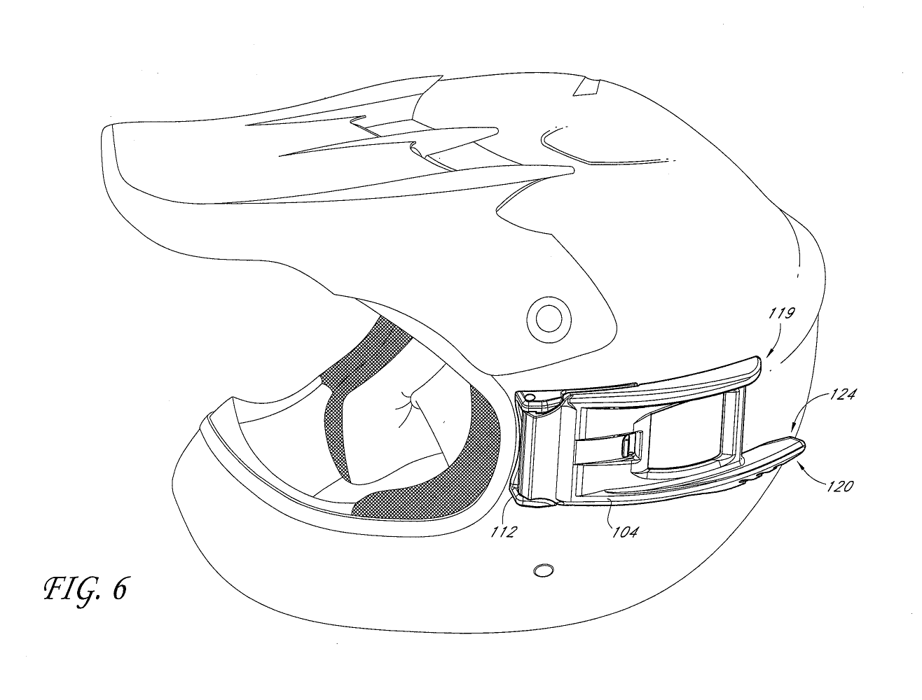

In some embodiments, such as the embodiment of FIGS. 2-6, the strap engagement member can comprise a thumb catch 120. The thumb catch 120 can comprise one or more projections 119. In some embodiments, the thumb catch 120 can comprise a projection 119 that is longer than another projection 119, as illustrated in the embodiment of FIGS. 2-6, for example. In some embodiments, such as the embodiment of FIGS. 2-6 for example, the thumb catch 120 can comprise an end 122 which is beveled such that when the strap engagement member is in the first position, the thumb catch 120 is adjacent, substantially adjacent, or close to the side 114 of the helmet 102 and the beveled end 122 of the thumb catch 120 forms a recess or an opening 124 (FIG. 6) which can accommodate at least a portion of a thumb or finger of the wearer to facilitate actuation or movement of the strap engagement member between the first position and the second position. In some embodiments, the opening 124 can accommodate at least a portion of a gloved thumb or finger. In some embodiments, the thumb catch 120 can comprise an aperture, a textured surface for increased friction, or both. The thumb catch 120 can be positioned at or near the second end 108 of the arm 104 (as illustrated, for example), at or near the first end 106 of the arm 104, or at a location between the first end 106 and the second end 108.

When the strap engagement member is in the first positioned, the strap engagement member preferably allows or facilitates placement of the goggles against the face of the wear so as to provide a comfortable and effective fit. In some embodiments, the strap engagement member can extend generally along the side 114 of the helmet 102 when in the first position, as shown in FIG. 6, for example. In some embodiments, the arm 104, including the first end 106 and the second end 108, can be in close proximity to the side 114 of the helmet 102 when in the first position, as shown in FIG. 6, for example. In some embodiments, the strap engagement member when in the first position can be located entirely rearward of an opening in the helmet 102 which accommodates the wearer's face, as illustrated, for example, in FIG. 6.

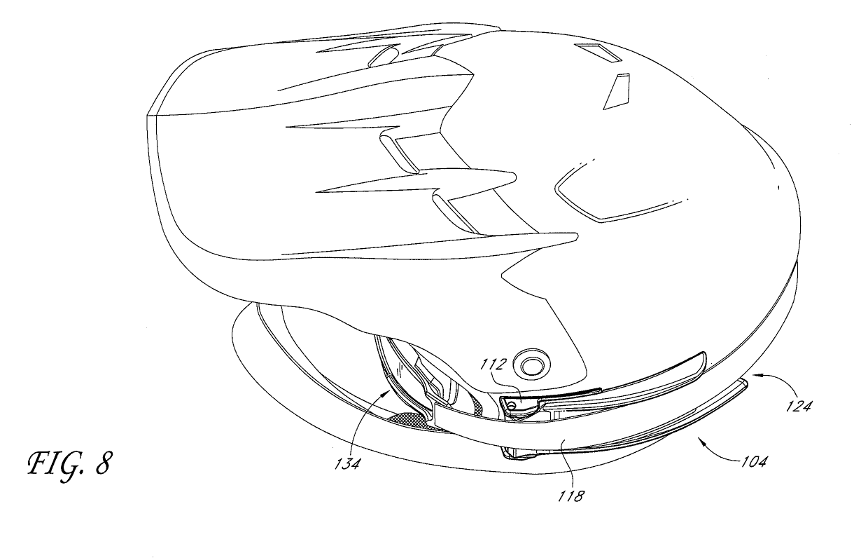

In some embodiments, such as the illustrated embodiments, for example, the strap 118 can hold the strap engagement member of the goggles position adjustment assembly 100 in the first or closed position, as shown in FIG. 8, so that the arm 104 cannot inadvertently move to the second position, shown in FIG. 1. In some embodiments such as those illustrated for example, the strap 118 of the goggles 134 can urge at least a portion of the strap engagement member, such as the arm 104, toward the head of the wear such that the strap engagement member moves toward either the first position or the second position when on a corresponding side of a stability inflection point.

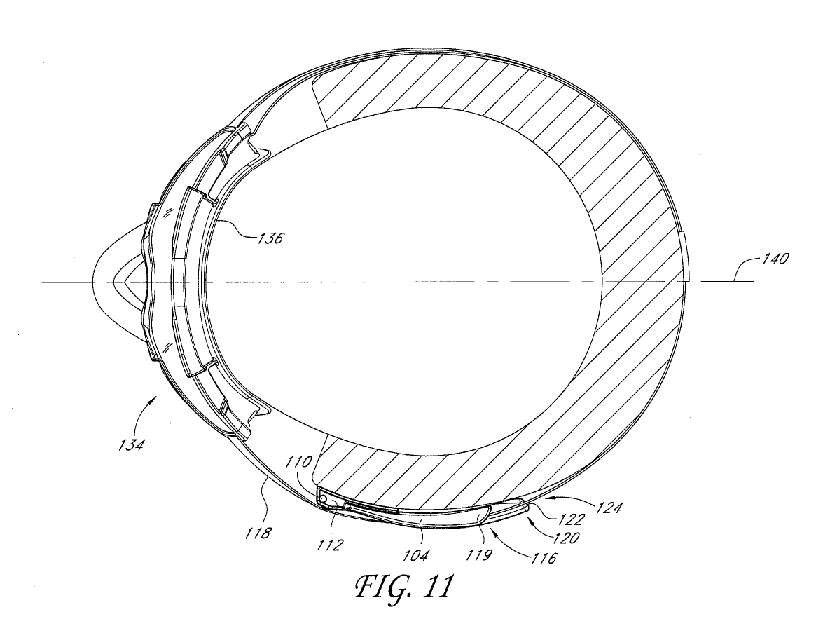

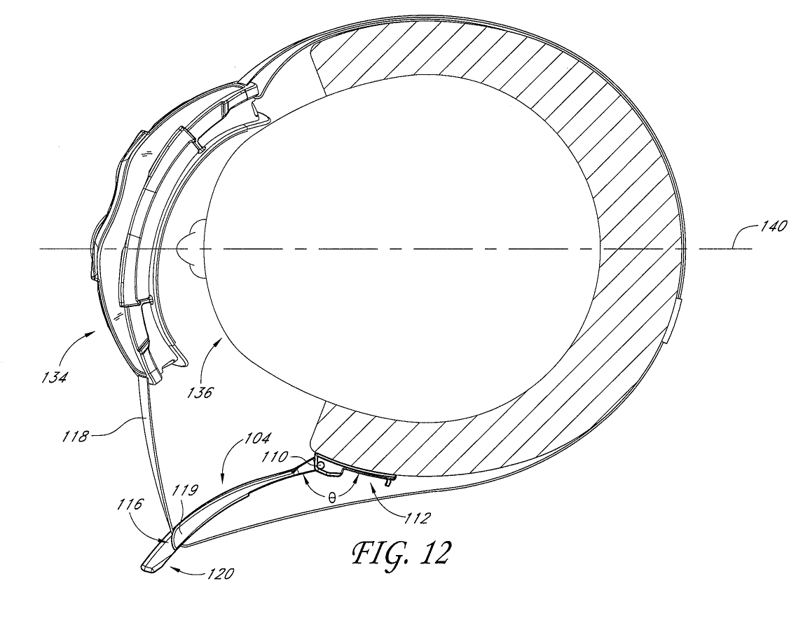

In some embodiments, when the strap engagement member is in the second position, shown in FIG. 1, at least a portion of the strap engagement member can be positioned forward of a rear-most portion of a perimeter of the face opening of the helmet 102, as illustrated for example in FIGS. 1, 7, 10, and 12. In some embodiments, when the goggles are in the second position, at least a portion of the strap engagement member can be positioned forward of the location of a rearmost attachment point of the strap 118 to the goggles when the goggles are pressed against the wearer's face, for example, as can be observed by a comparison of FIGS. 11 and 12. In some embodiments, such as the illustrated embodiments, for example, the at least a portion of the strap engagement member that is advanced forward can comprise the second end 108 of the arm 104. In some embodiments, such as those illustrated in FIG. 11-13, when in the second position at least a portion of the strap engagement member can be located forward of pivot axis or a coupling about which the strap engagement moves.

In some embodiments, in addition or alternative to the above-described forward motion, the at least a portion of the strap engagement member, e.g. the second end 108 of the arm 104, can be located farther from a plane 140 (FIGS. 11 and 12) that longitudinally bisects the helmet 102 between the left and right sides, from a mount (if present) or from a coupling to a helmet, than it is in the first position. In some such embodiments, the position of the strap engagement member in the second position can advantageously hold the strap engagement member in the second, advanced or open position, such that the assembly 100 remains in the second, advanced or open position, as shown in FIG. 1, until affirmatively moved to the first, closed or retracted position. For example, in some embodiments, such as those illustrated for example, the strap 118 of the goggles 134 may need to be stretched in the process of moving the strap engagement member between the first position and the second position.

Preferably, when in the second position, the assembly 100 holds the goggles 134 away from the wearer's face 136 by a distance that is sufficient to allow air to flow between the wearer's face and the goggles. The separation between the goggles 134 and the wearer's face 136 may be greater at one side of the goggles than the other. As illustrated in FIG. 12, one side of the goggles may be spaced from the face 136 while the other side of the goggle remains in contact, or nearly in contact, with the face 160. The separation between the face 136 and the goggles 134 can be measured by the distance between the face and goggles at a point of greatest separation between them or as the distance between the goggles and the face at a location closest to the strap engagement member. The distance between the goggles and the wearer's face may be, for example, at least ¼ inch, at least ½ inch, at least 1 inch, at least 1.5 inches, at least 2 inches, at least 2.5 inches, or at least 3 inches.

In some embodiments, when in the second position, the second end 108 of the arm 104 can be located forward of the hinge 110 (if present), the rearward-most portion of the face opening of the helmet 102, the rearmost point if attachment of the strap 118 to the goggles when pressed against the face, or a combination of those. In some embodiments, the second end of the arm 104 can be located between one (1) and four (4) inches forward of at least one of those reference points when in the second position. In some embodiments, the second end of the arm 104 can be located two (2) to three (3) inches forward of at least one of those reference points when in the second position.

In some embodiments, the goggles position adjustment assembly 100 can comprise a stop 126 to limit the movement of the strap engagement member relative to the helmet 102. For example, FIG. 3 illustrates one embodiment of a stop 126, which comprises a projection located at or near the first end 106 of the arm 104. The stop 126 is sized and positioned such that forward movement of the arm is limited to the location shown in FIG. 1. In some embodiments, the stop 126 defines a surface which cooperates with a surface on the mounting plate 112 to limit forward movement of the arm 104 and, preferably, rotational movement of the arm 104 about the axis of the hinge 110 (if present). The stop can be integrated with the hinge or other connector in some embodiments. In some embodiments, the stop can be spaced farther from the hinge or connector than shown in the drawings.

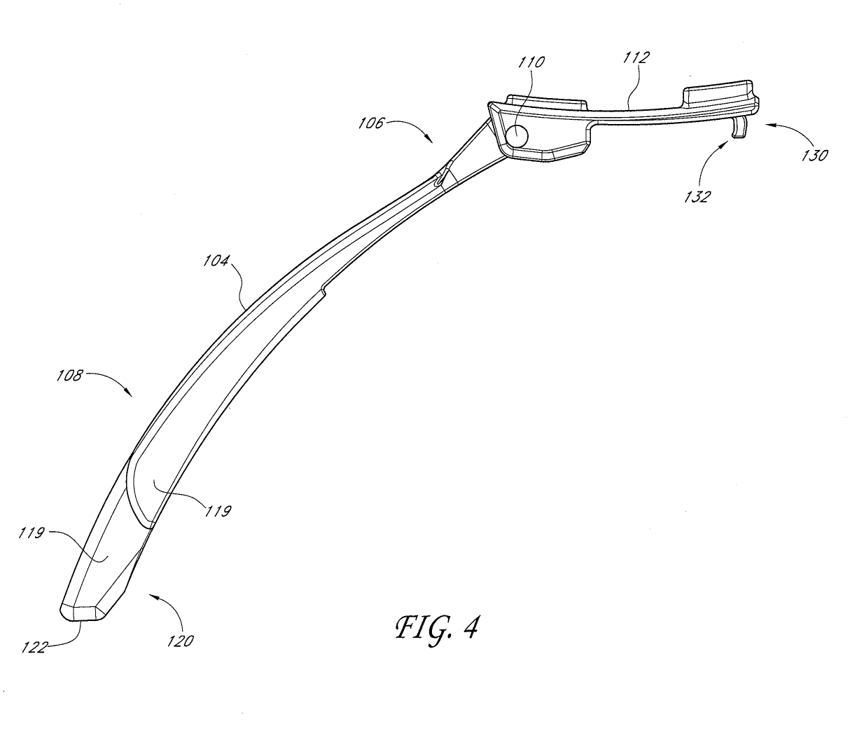

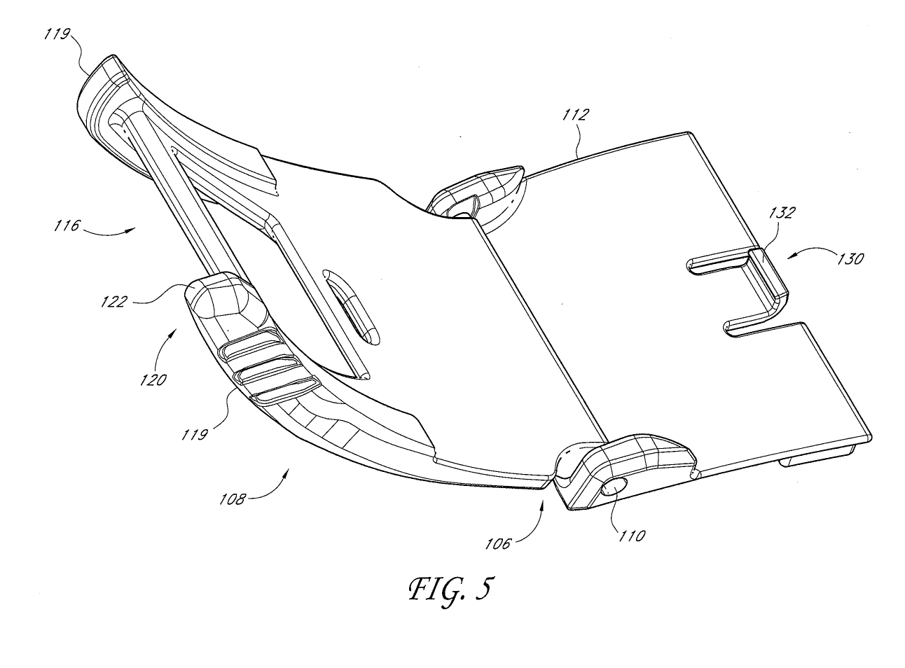

In some embodiments, the goggles position adjustment assembly 100 can comprise a mechanism for holding the strap engagement member in the first position in cooperation with or independent of the influence of the strap 118. For example, in some embodiments, such a mechanism comprises a catch, clasp, lock or similar device for releasably holding the strap engagement member in the first position. FIGS. 3 through 5 illustrate an exemplifying embodiment of such a mechanism. As illustrated in FIGS. 3-5, for example, the arm 104 can comprise an opening or recess 128 and the mounting plate 112 can comprise a complementary projection 130 shaped to interfere with a perimeter or side of the opening or recess 128, as shown in FIG. 5. In particular, the projection can comprise an end 132 that is shaped to engage perimeter or side of the opening or recess 128 to prevent the arm 104 from moving away from the mounting plate 112, for example as illustrated in FIGS. 4 and 5. In some embodiments, the opening or recess 128 can be located on the mounting plate 112 and the arm 104 can comprise the complimentary structure. In embodiments in which the strap engagement member is attached directly to a helmet, the helmet can comprise one of a projection and an opening or recess 128, while the strap engagement member comprises the other of a projection and an opening or a recess 128.

FIG. 13 illustrates an exemplifying embodiment wherein a strap engagement member 100′ is attached directly to a helmet 102′. The embodiment of FIG. 13 is similar to that of FIG. 12 in many respects, thus similar reference numerals have been used in connection with similar components with the addition of a prime 0 thereafter. As illustrated in FIG. 13, the helmet 102′ can comprise a recess 138′ configured to receive some or all of the strap engagement member 100′ when in the first position. Preferably, when the strap engagement member is in the first position the thumb catch 120′ remains accessible to the wearer such as providing a gap or space between the thumb catch 120′ and the helmet 102′ sufficiently large to receive at least a portion of a thumb or finger of the wearer.

Although the invention has been disclosed in the context of certain specific embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of the invention, will be readily apparent to those of skill in the art based upon this disclosure. It is also contemplated that the various combinations or subcombinations of the specific features and aspects of the embodiments may be made and still fall within the scope of the invention. It should be understood that various features and aspects of the disclosed embodiments can be combined with or substituted for one another in order to form varying modes of the disclosed invention. For example, various options, alternatives, and variations have been described in connection with, for example, the configuration of the arm 104, 104′, the hinge 110, 110′, the mounting plate 112, the recess 116 and other strap retaining features, the thumb/finger catch 120, 120′, the first and second positions of the strap engagement member, the stop 126, and mechanisms for holding the strap engagement member in the first position. These features are specifically contemplated as being combined together in any combination, including combinations excluding one or more of these features, so long as those features are not incompatible. Thus, it is intended that the scope of at least some of the embodiments of the present invention herein described should not be limited by the particular disclosed embodiments described above. Also, nothing in the foregoing description should be construed to imply that any particular feature or component is essential to the invention.

What is claimed is:

1. An assembly for selective movement of a goggles relative to a face of a wearer of a helmet, the helmet having an opening for the face of the wearer, the assembly comprising:
a strap engagement member configured to be movably mounted to the helmet for movement between a first position and a second position, the strap engagement member being configured to be in close proximity to an external surface of the helmet in the first position, and at least a portion of the strap engagement member being located forward of the face opening by distance sufficient to hold a goggles away from the face of the wearer in the second position.

2. The assembly of claim 1, wherein the strap engagement member retains the strap.

3. The assembly of claim 1, wherein the at least a portion of the strap engagement member is located between approximately 1 and 4 inches forward of a rearmost boundary of the opening for the face in the second position.

4. The assembly of claim 3, wherein the at least a portion of the strap engagement member is located between approximately 2 and 3 inches forward of a rearmost boundary of the opening for the face in the second position.

5. The assembly of claim 1, wherein the at least a portion of the strap engagement member is located farther from a plane that longitudinally bisects the helmet in the second position than in the first position.

6. The assembly of claim 1, wherein the strap engagement member is connected to the helmet by a hinge, and the strap engagement member pivots about the hinge between the first position in the second position.

7. The assembly of claim 6, further comprising a stop member to restrict the range of motion of the strap engagement member about the hinge.

8. The assembly of claim 1, further comprising a base plate.

9. The assembly of claim 8, further comprising an adhesive for attachment of the base plate to the helmet.

10. The assembly of claim 1, further comprising a thumb catch.

11. The assembly of claim 1, wherein the strap engagement member is configured to be mounted to the left side of a helmet.

12. The assembly of claim 1, further comprising a catch for holding the strap engagement member in the first position.

13. The assembly of claim 1, further comprising a helmet.

14. A method of adjusting the position of a face mask relative to a face of a wearer, comprising the steps of:
attaching an assembly to a helmet; and
moving at least a portion of the assembly from a first position, which permits the face mask to substantially seal against the face of the wearer, to a second position, in which at least a portion of the face mask is spaced from the face of the wearer.

15. The method of claim 14, wherein said moving step comprises moving the second position forward of the first position.

16. The method of claim 14, wherein said moving step comprises moving said portion of the assembly farther from a plane that longitudinally bisects the helmet than when that portion is the first position.

17. The method of claim 16, wherein the helmet comprises an opening in a forward portion to accommodate at least a portion of the face, and wherein said moving step comprises moving said portion of the assembly both forward of the first position and farther from a plane that longitudinally bisects the helmet to the second position from the first position.
